(19) United States
(12) Patent Application Publication
(10) Pub. No.: US 2005/0022827 A1
(43) Pub. Date: Feb. 3, 2005

(54) Method and device for gastrointestinal bypass

(76) Inventors: Sang Hoon Woo, Durham, NC (US); Amrish Jayprakash Walke, Menlo Park, CA (US)

Correspondence Address: SANG HOON WOO, 206 OLD DOCK TRAIL, CARY, NC, 27519, US

(21) Appl. No.: 10/885,209

(22) Filed: Jul. 6, 2004

Related U.S. Application Data
(63) Continuation of application No. 10/694,149, filed on Oct. 27, 2003.
(60) Provisional application No. 60/424,248, filed on Nov. 6, 2002.

(51) Int. Cl. A61B 19/00

(52) U.S. Cl. 128/898

(57) ABSTRACT

The present invention provides a device for causing weight loss in obese patients comprising an implant that creates a gastrointestinal bypass between a first region of the gastrointestinal tract and a second region of the gastrointestinal tract. In one embodiment, the implant comprises an adjustable opening to adjust the fraction of food material passing through the gastrointestinal bypass. Also disclosed is a method for causing weight loss in obese patients comprising the step of creating a gastrointestinal bypass between a first region of the gastrointestinal tract and a second region of the gastrointestinal tract.

This application is a continuation in part of U.S. patent application Ser. No. 10/694,149, filed Oct. 27, 2003, titled intestinal bypass device to treat obesity incorporated herein by reference that claims the priority of U.S. Provisional Patent Application No. 60/424,248, filed Nov. 06, 2002, titled device to treat obesity by intestinal bypass incorporated herein by reference.

### BACKGROUND OF THE INVENTION

The present invention relates to surgical devices to treat obesity. More particularly, the present invention relates to implants for causing weight loss.

Obesity is a serious health problem especially in developed countries. Approximately 60 million adults in the U.S. are obese. Obesity leads to several health problems such as increased risk of illness and death due to coronary artery disease, diabetes and stroke.

Obesity has high medical costs due to the high prevalence of obesity and the various health problems associated with it. In a study conducted in 1998, the direct medical costs due to obesity were estimated to be $51.64 billion in the US (Source: Website of the American Obesity Association). These costs could increase in the future as the prevalence of obesity is steadily increasing. In the United States, the percentage of children and adolescents who are obese has doubled in the last 20 years. Thus, there is an urgent need to treat this serious health problem.

Obesity is treated by reducing the patient's weight. Weight loss methods can be broadly divided into diet modification, exercise therapy, pharmacological therapy and surgical procedures.

Surgical procedures are generally used for weight loss when diet modification, exercise therapy and pharmacological therapy fail to cause required weight loss. The most commonly used surgical procedures for weight loss are Roux-en-Y gastric bypass procedures, restrictive gastric operations, malabsorptive operations such as biliopancreatic diversion and intestinal bypass procedures. The Roux-en-Y gastric bypass procedure involves creating a stomach pouch out of a small portion of the stomach and attaching it directly to the small intestine, bypassing a large part of the stomach and duodenum. The small stomach pouch holds much smaller amounts of food at a time, and hence the patient experiences a feeling of satiety even after eating a small quantity of food. Also, fat absorption from food is substantially reduced as the food bypasses a large portion of the duodenum.

Restrictive gastric operations cause weight loss by restricting the food intake by the patient. A portion of the stomach is surgically modified to form a small pouch. The food enters the pouch from the esophagus. The outlet from the pouch to the rest of the stomach is restricted. This restriction delays the emptying of food from the pouch, causing a feeling of fullness even after consuming small amounts of food.

Another type of restrictive procedure is called LAP-BAND™. In this procedure, an inflatable silicone band is fastened around the upper stomach to create a new, stomach pouch. This limits the amount of food the patient can eat which in turn leads to weight loss.

Malabsorptive operations such as biliopancreatic diversion cause weight loss by restricting the food intake and also by reducing the fraction of calories absorbed by the body from the digested food. In a biliopancreatic diversion, portions of the stomach are removed along with the duodenum and the jejunum. This reduces the fraction of calories absorbed from the digested food, thereby causing weight loss.

Conventional intestinal bypass procedures cause weight loss by removing a section of the small intestine and reconnecting the remaining sections of the small intestine. In some cases, devices called anastomosis devices are used to reconnect the remaining sections of the small intestine. Removal of a section of the small intestine reduces the effective length of the intestine. As the intestine is the main site of absorption of nutrients from food material, reducing the effective length of the intestine reduces the amount of nutrients that are absorbed by the body from the food. This leads to weight loss.

The abovementioned surgical procedures are invasive and require major modifications to the patient's anatomy. Further, the anatomical modifications due to these procedures cannot be frequently adjusted to adjust the rate of weight loss. For example, the anastomosis devices used during conventional intestinal bypass procedures cannot be frequently adjusted. Further, procedures like LAP-BAND™ require significant behavior modifications by the patient. Also, if these surgical procedures cause severe side effects to the patient, the anatomical modifications cannot be easily reversed.

Thus, there is a need for an obesity treatment that does not need significant modifications to the patient's anatomy. Further, there is a need for an obesity treatment whose parameters can be adjusted frequently to adjust the rate of weight loss. Further, there is a need for an obesity treatment whose parameters can be adjusted with minimal discomfort to the patient. Further, there is a need for an obesity treatment that does not require significant behavior modification by the patient. Further, there is a need for an obesity treatment that can be reversed if the patient experiences significant side effects.

### BRIEF SUMMARY OF THE INVENTION

An object of the present invention is to provide an obesity treatment whose parameters can be adjusted to adjust the rate of weight loss. Another object of the present invention is to provide an obesity treatment whose parameters can be adjusted with minimal discomfort to the patient. Another object of the present invention is to provide an obesity treatment that does not require significant behavior modification by the patient. Another object of the present invention is to provide an obesity treatment that does not cause significant permanent modifications to the patient's anatomy.

To achieve the foregoing objects, and in accordance with the purpose of the present invention, the present invention provides a device for causing weight loss in obese patients comprising an implant that creates a gastrointestinal bypass between a first region of the gastrointestinal tract and a second region of the gastrointestinal tract. A part of food material passing through the gastrointestinal tract from the first region of the gastrointestinal tract to the second region of the gastrointestinal tract is diverted through the gastrointestinal bypass. Diversion of a part of food material through the gastrointestinal bypass causes a reduction in the total nutrients absorbed by the body from the food material. This causes the patient to lose weight. In one embodiment, the implant comprises an adjustable opening to adjust the fraction of food material passing through the gastrointestinal bypass and hence adjust the rate of weight loss.

The present invention also provides a method for causing weight loss in obese patients comprising the steps of creating a gastrointestinal bypass between a first region of the gastrointestinal tract and a second region of the gastrointestinal tract. In one embodiment, the method for causing weight loss in obese patients comprises the steps of creating a gastrointestinal bypass between a first region of the gastrointestinal tract and a second region of the gastrointestinal tract with an adjustable opening and adjusting the size of the adjustable opening to adjust the patient's weight loss.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 illustrates the general working principle of the invention;

FIG. 2 illustrates an example of a method of treating a patient using the invention;

FIG. 3 illustrates a second example of a method of treating a patient using the invention;

FIG. 4 illustrates a third example of a method of treating a patient using the invention;

FIG. 5 illustrates a fourth example of a method of treating a patient using the invention;

FIG. 6 illustrates a fifth example of a method of treating a patient using the invention;

FIG. 7 illustrates a sixth example of a method of treating a patient using the invention;

FIG. 8 illustrates a seventh example of a method of treating a patient using the invention, the method being an improvement of the existing Roux-en-Y gastric bypass procedure;

FIGS. 9A and 9B illustrate an embodiment of the device of the invention;

FIGS. 10A and 10B illustrate a second embodiment of the device of the invention;

FIGS. 11A and 11B illustrate a sectional view of an adjustable connection made using the embodiment illustrated in FIGS. 9A and 9B;

FIGS. 12A and 12B illustrate a third embodiment of the device of the invention;

FIGS. 13A and 13B illustrate a sectional view of an adjustable connection made using the embodiment illustrated in FIGS. 12A and 12B;

FIGS. 14A and 14B illustrate a fourth embodiment of the device of the invention;

FIGS. 15A and 15B illustrate a sectional view of an adjustable connection made using the embodiment illustrated in FIGS. 14A and 14B;

FIG. 16 illustrates an embodiment of a mechanism to adjust the device illustrated in FIGS. 14A and 14B;

FIG. 17 illustrates a sectional view of a fifth embodiment of the device of the invention;

FIG. 18 illustrates a sixth embodiment of the device of the invention;

FIG. 19 illustrates a seventh embodiment of the device of the invention;

FIG. 20 illustrates a sectional view of an eighth embodiment of the device of the invention;

FIG. 21 illustrates a sectional view of a ninth embodiment of the device of the invention;

FIG. 22 illustrates the steps of the present invention to achieve weight loss in patients.

### DETAILED DESCRIPTION OF THE INVENTION

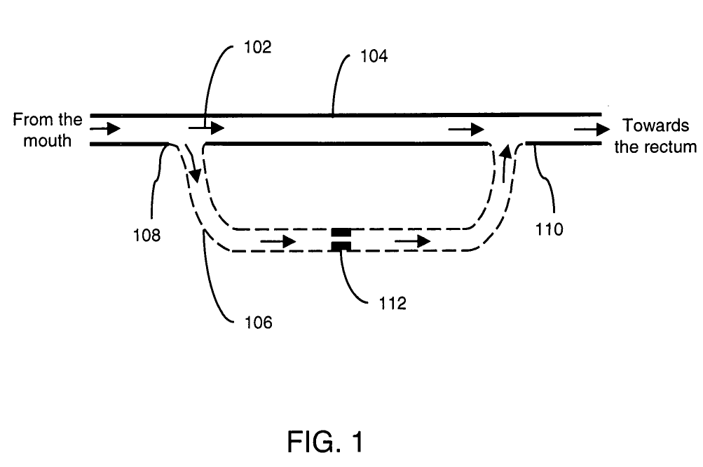

FIG. 1 illustrates the general working principle of the invention. Food material 102 ingested through the mouth enters gastrointestinal tract 104. In gastrointestinal tract 104, food material 102 undergoes a process called digestion. In the digestion process, food material 102 is converted to a form that can be easily absorbed by the body. Food material 102 then undergoes a process known as absorption, wherein digested food material 102 is absorbed by the body. Ultimately, a fraction of food material 102 is absorbed by the body. Creation of a gastrointestinal bypass 106 between a first region 108 of the gastrointestinal tract and a second region 110 of the gastrointestinal tract causes a fraction of food material 102 to flow through gastrointestinal bypass 106. This changes the time food material 102 resides in gastrointestinal tract 104 which in turn leads to a decrease in digestion or absorption or both. Ultimately, this leads to a smaller fraction of food material 102 being absorbed by the body and hence leads to weight loss. The fraction of food material 102 flowing through gastrointestinal bypass 106 can be controlled by an adjustable opening 112. Enlarging adjustable opening 112 causes a greater portion of food material 102 to flow through gastrointestinal bypass 106. This reduces the fraction of ingested food that is absorbed by the body which in turn increases the rate of weight loss. Similarly, reducing the size of adjustable opening 112 increases the fraction of ingested food that is absorbed by the body which in turn reduces the rate of weight loss. In this manner, the rate of weight loss can be adjusted to maintain it at a desired level.

In this document, unless specified, intestine can mean either small intestine or large intestine.

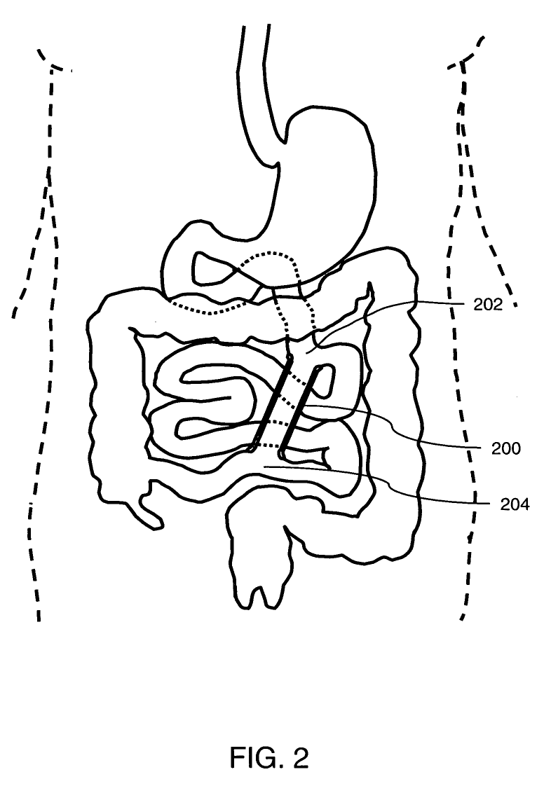

FIG. 2 illustrates an example of a method of treating a patient using the invention. In this method, an implant 200 is provided that connects a first region 202 of the intestine to a second region 204 of the intestine to create an intestinal bypass. First region 202 is located on the small intestine. Second region 204 is located downstream from first region 202. Second region 204 can be located on the small intestine or the large intestine. This method achieves weight loss by reducing the amount of food material that is absorbed by the body. A portion of food material flowing through the intestine from the first region 202 to the second region 204 is diverted through implant 200. As the intestine is the main site for absorption of the food material, the portion of food material flowing through implant 200 is less absorbed by the body. This causes the patient to lose weight.

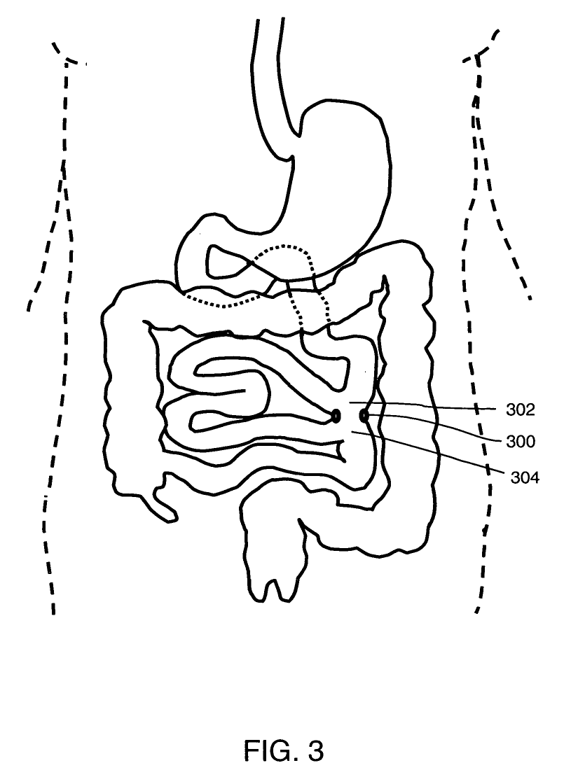

FIG. 3 illustrates a second example of a method of treating a patient using the invention. The invention comprises a ring shaped implant 300 that connects a first region 302 of the intestine to a second region 304 of the intestine to create an intestinal bypass.

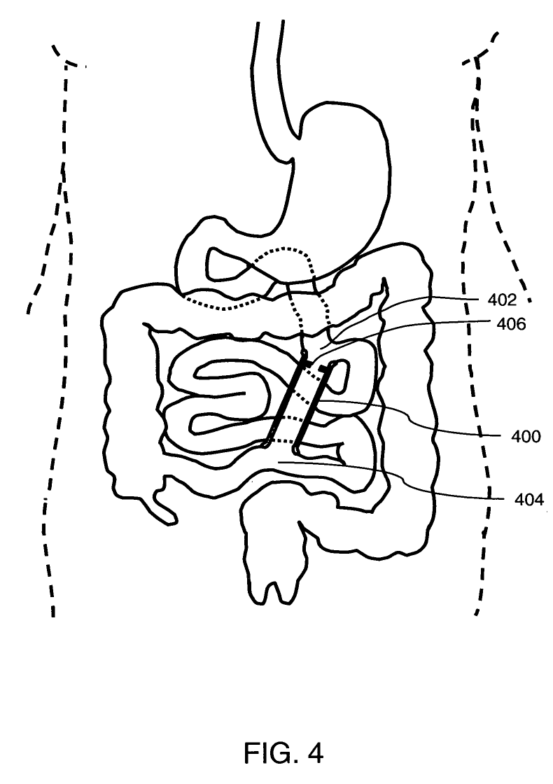

FIG. 4 illustrates a third example of a method of treating a patient using the invention. The invention comprises a tubular implant 400 that connects a first region 402 of the intestine to a second region 404 of the intestine to create an intestinal bypass. Tubular implant 400 comprises an adjustable opening 406 to adjust the rate of weight loss. The rate of weight loss can be controlled by adjusting the size of adjustable opening 406. Increasing the size of adjustable opening 406 will cause a greater portion of the food material to pass through the intestinal bypass. This will reduce the amount of food material absorbed by the intestine and thus increase the rate of weight loss. Similarly, reducing the size of adjustable opening 406 will reduce the rate of weight loss.

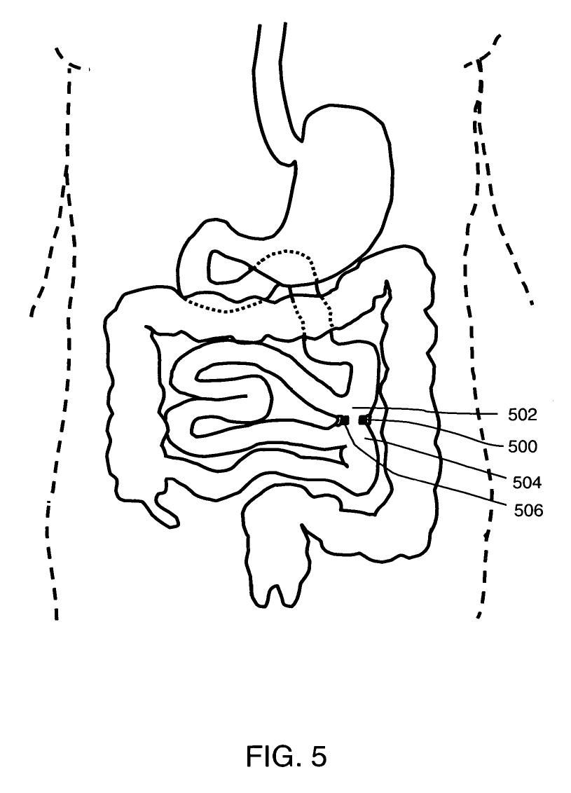

FIG. 5 illustrates a fourth example of a method of treating a patient using the invention. The invention comprises a ring shaped implant 500 that connects a first region 502 of the intestine to a second region 504 of the intestine to create an intestinal bypass. Ring shaped implant 500 comprises an adjustable opening 506 to adjust the rate of weight loss. The rate of weight loss can be controlled by adjusting the size of adjustable opening 506. Increasing the size of adjustable opening 506 will cause a greater portion of the food material to pass through the intestinal bypass. This will reduce the amount of food material absorbed by the intestine and thus increase the rate of weight loss. Similarly, reducing the size of adjustable opening 506 will reduce the rate of weight loss.

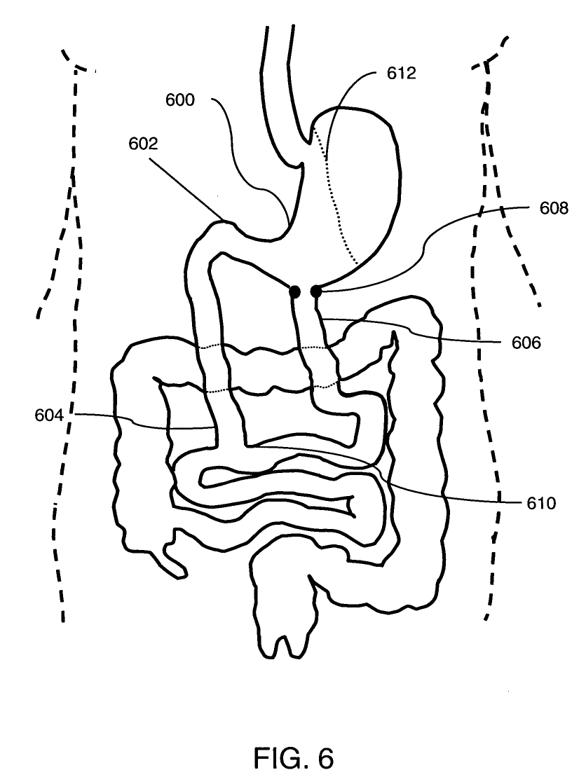

FIG. 6 illustrates a fifth example of a method of treating a patient using the invention. In the normal anatomy, stomach 600 is connected to duodenum 602 that continues as small intestine which ultimately continues as large intestine. In the method illustrated in FIG. 6, the small intestine is cut between a first region 604 of small intestine and a second region 606 of small intestine. Second region 606 of small intestine is then anastomosed to a region of stomach 600 by an end-to-side anastomosis (or side-to-side anastomosis). An adjustable opening 608 is provided near the anastomosis of stomach 600 and second region 606 of small intestine. Thereafter, first region 604 of small intestine is anastomosed to a distal region 610 of small intestine by an end-to-side anastomosis. Distal region 610 is located distal to second region 606 of small intestine as illustrated in FIG. 6. Food material flowing through stomach 600 is divided into two parts: first part flowing through duodenum 602 and second part flowing through adjustable opening 608. Thus, food material bypasses sections of the gastrointestinal tract. This reduces the total amount of food material that is absorbed by the body which in turn leads to weight loss. The rate of weight loss can be adjusted by adjusting the size of adjustable opening 608. As an optional step, the volume of stomach 600 can be reduced. One way to achieve this is by using staples 612 to isolate a volume of stomach 600.

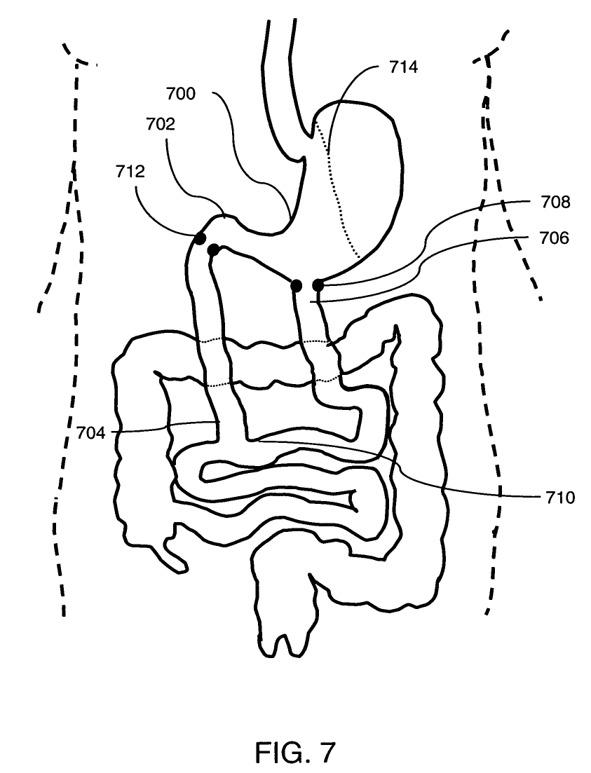

FIG. 7 illustrates a sixth example of a method of treating a patient using the invention. The illustrated method is similar to the method illustrated in FIG. 6. In the normal anatomy, stomach 700 is connected to duodenum 702 that continues as small intestine which ultimately continues as large intestine. In the method illustrated in FIG. 7, the small intestine is cut between a first region 704 of small intestine and a second region 706 of small intestine. Second region 706 of small intestine is then anastomosed to a region of stomach 700 by an end-to-side anastomosis. A first adjustable opening 708 is provided near the anastomosis of stomach 700 and second region 706 of small intestine. Thereafter, first region 704 of small intestine is anastomosed to a distal region 710 of small intestine by an end-to-side anastomosis. Distal region 710 is located distal to second region 706 of small intestine as illustrated in FIG. 7. Thereafter, a second adjustable opening 712 is provided near duodenum 702. Food material flowing through stomach 700 is divided into two parts: first part flowing through duodenum 702 and second part flowing through first adjustable opening 708. Thus, food material bypasses sections of the gastrointestinal tract. This reduces the total amount of food material that is absorbed by the body which in turn leads to weight loss. The rate of weight loss can be adjusted by adjusting the size of first adjustable opening 708 and second adjustable opening 712. As an optional step, the volume of stomach 700 can be reduced. One way to achieve this is by using staples 714 to isolate a volume of stomach 700.

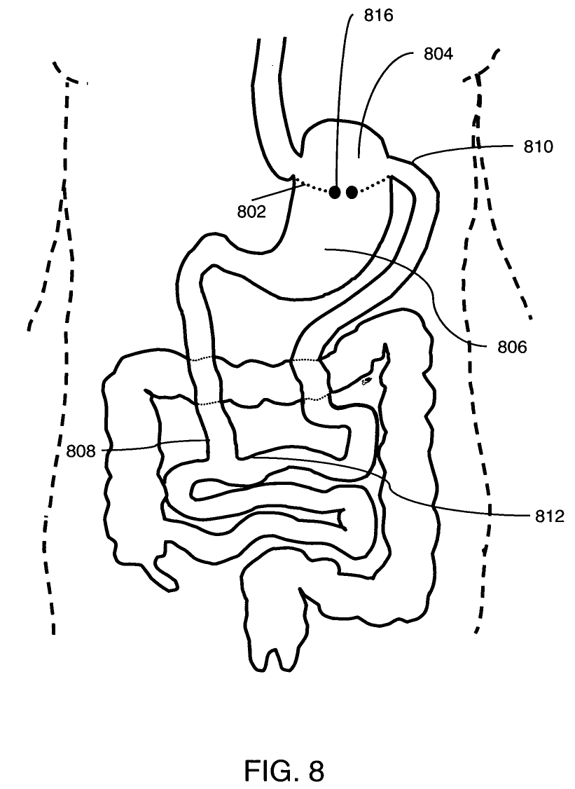

FIG. 8 illustrates a seventh example of a method of treating a patient using the invention, the method being an improvement of the existing Roux-en-Y gastric bypass procedure. In the Roux-en-Y procedure, a series of staples 802 divide the stomach into two regions: a first region 804 of stomach and a second region 806 of stomach. Thereafter, the small intestine is cut between a first region 808 of small intestine and a second region 810 of small intestine. Second region 810 of small intestine is then anastomosed to first region 804 of stomach by an end-to-side anastomosis. Thereafter, first region 808 of small intestine is anastomosed to a distal region 812 of small intestine by an end-to-side anastomosis. Distal region 812 is located distal to second region 810 of small intestine as illustrated in FIG. 8. In the improved Roux-en-Y procedure of the invention, an adjustable opening 816 is provided between first region 804 of stomach and second region 806 of stomach. Thus, food material entering first region 804 of stomach is divided into two parts: a first part flowing out through second region 810 of small intestine and a second part flowing out through adjustable opening 816. The rate of weight loss can be adjusted by adjusting the size of adjustable opening 816.

The abovementioned methods can be used with open surgical, laparoscopic, endoscopic or interventional procedures.

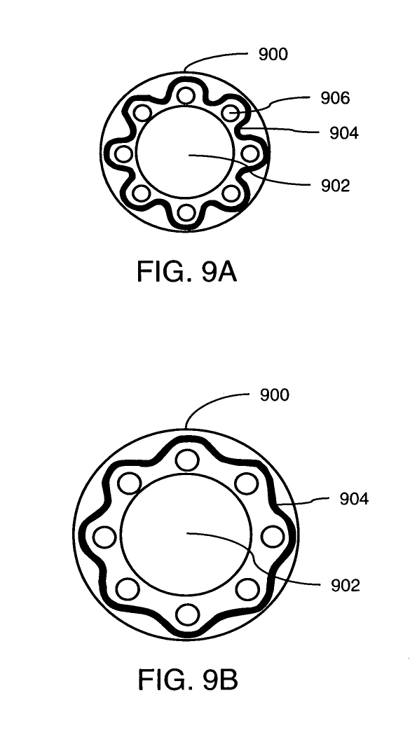

FIGS. 9A and 9B illustrate an embodiment of a device to create an adjustable opening. In this embodiment, a deformable implant 900 is provided. Deformable implant 900 is substantially ring shaped and encloses a lumen 902. Deformable implant 900 comprises a deformable element 904. In one embodiment, deformable element 904 is in the form of a stiff metal loop of sufficient stiffness so that deformable implant 900 is substantially undeformable under the normal physiological forces acting on it after implantation. In another embodiment, deformable element 904 is in the form of a deformable stent of sufficient stiffness so that deformable implant 900 is substantially undeformable under the normal physiological forces acting on it after implantation. Size of lumen 902 can be adjusted by dilating or contracting deformable implant 900. One example of a method to contract lumen 902 is laparoscopic compression of deformable implant 900 by a device inserted in the peritoneum through a small incision in the abdominal wall. One example of a method to dilate lumen 902 is endoscopic dilation of deformable implant 900 by a dilating device inserted endoscopically in the gastrointestinal tract. The dilating device may be inserted in the gastrointestinal tract through a trans-oral, trans-nasal or trans-anal approach. Deformable implant 900 further comprises apertures 906. Apertures 906 facilitate the creation of anastomosis between regions of the gastrointestinal tract that are being connected by deformable implant 900. In one embodiment, parts of deformable implant 900 except deformable element 904 are biofragmentable or bioabsorbable. FIG. 9A illustrates deformable implant 900 in a contracted state. FIG. 9B illustrates deformable implant 900 in a dilated state.

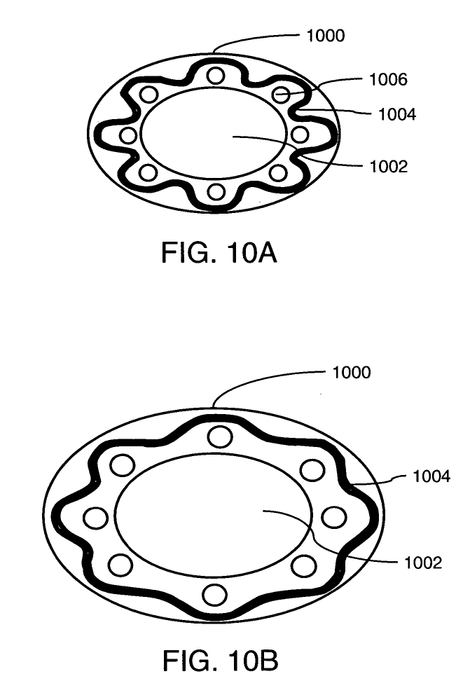

FIG. 10A and FIG. 10B illustrate a second embodiment of the invention. In this embodiment, a deformable implant 1000 is provided. Deformable implant 1000 encloses a lumen 1002. Lumen 1002 is substantially elongated in one direction. In one embodiment, deformable implant 1000 is substantially rectangular in cross-section. Deformable implant 1000 comprises a deformable element 1004. In one embodiment, deformable element 1004 is in the form of a stiff metal loop of sufficient stiffness so that deformable implant 1000 is substantially undeformable under the normal physiological forces acting on it after implantation. In another embodiment, deformable element 1004 is in the form of a deformable stent of sufficient stiffness so that deformable implant 1000 is substantially undeformable under the normal physiological forces acting on it after implantation. Size of lumen 1002 can be adjusted by dilating or contracting deformable implant 1000. One example of a method to contract lumen 1002 is laparoscopic compression of deformable implant 1000 by a device inserted in the peritoneum through a small incision in the abdominal wall. One example of a method to dilate lumen 1002 is endoscopic dilation of deformable implant 1000 by a dilating device inserted endoscopically in the gastrointestinal tract. The dilating device may be inserted in the gastrointestinal tract through a trans-oral, trans-nasal or trans-anal approach. Deformable implant 1000 further comprises apertures 1006. Apertures 1006 facilitate the creation of anastomosis between regions of the gastrointestinal tract that are being connected by deformable implant 1000. In one embodiment, parts of deformable implant 1000 except deformable element 1004 are biofragmentable or bioabsorbable. FIG. 9A illustrates deformable implant 1000 in a contracted state. FIG. 9B illustrates deformable implant 1000 in a dilated state.

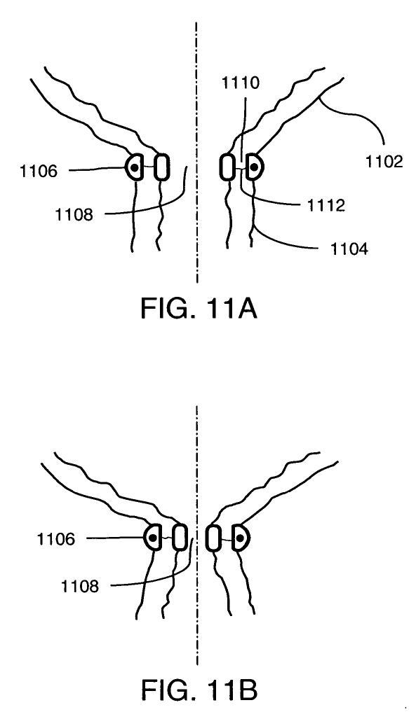

FIGS. 11A and 11B illustrate a sectional view of an adjustable connection made using the embodiment illustrated in FIGS. 9A and 9B. The adjustable connection is created between a first region 1102 of the gastrointestinal tract and a second region 1104 of the gastrointestinal tract. The adjustable connection is created using a deformable implant 1106 that encloses a lumen 1108. The size of lumen 1108 is adjusted by deformation of deformable implant 1106. Deformable implant 1106 further comprises apertures 1110. Regions of first region 1102 of the gastrointestinal tract and second region 1104 of the gastrointestinal tract come into physical contact with each other through apertures 1110. This facilitates the two regions of the gastrointestinal tract to fuse with each other through apertures 1110. This enables the creation of a stable anastomosis 1112 between first region 1102 of the gastrointestinal tract and second region 1104 of the gastrointestinal tract. FIGS. 11A and 11B illustrate sectional views of the adjustable connection when deformable implant 1106 is in a dilated state and a contracted state respectively.

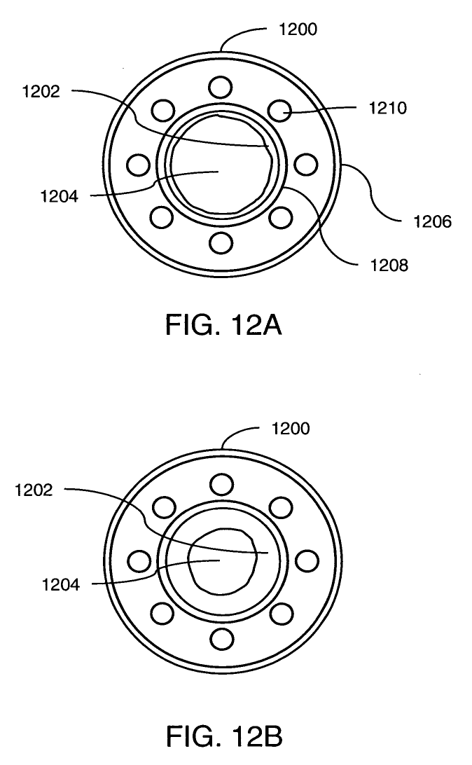

FIGS. 12A and 12B illustrate a third embodiment of a device to create an adjustable opening. In this embodiment, an adjustable implant 1200 is provided. Adjustable implant 1200 is substantially ring shaped and comprises an inflatable member 1202. Inflatable member 1202 encloses a lumen 1204. Inflatable member 1202 can be inflated or deflated to adjust the diameter of lumen 1204. Adjustable implant 1200 further comprises a first circular flange 1206 and a second circular flange 1208. First circular flange 1206 is located around the periphery of adjustable implant 1200 and second circular flange 1208 is located around lumen 1204. First circular flange 1206 and second circular flange 1208 are present on both sides of adjustable implant 1200. First circular flange 1206 and second circular flange 1208 are of a height sufficient to enclose an annular cavity of a depth sufficient to receive a region of the gastrointestinal tract. Adjustable implant 1200 further comprises apertures 1210. Apertures 1210 enable regions of the gastrointestinal tract to come into physical contact with each other. This facilitates the regions of the gastrointestinal tract to fuse with each other to create a stable anastomosis. FIG. 12A illustrates adjustable implant 1200 in which inflatable member 1202 is deflated. FIG. 12B illustrates adjustable implant 1200 in which inflatable member 1202 is inflated

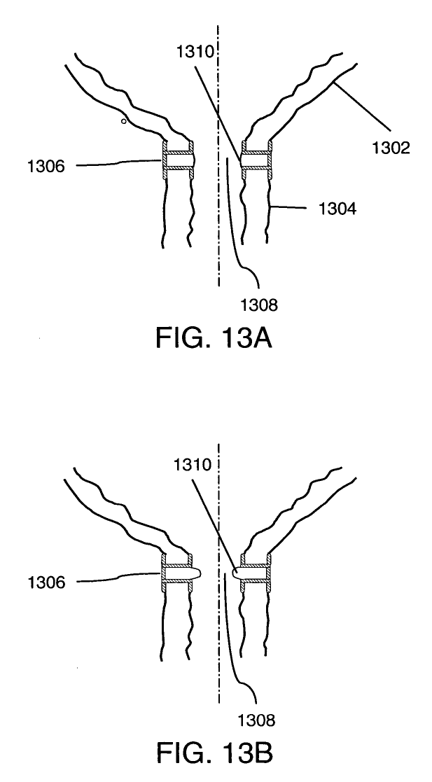

FIGS. 13A and 13B illustrate a sectional view of an adjustable connection made using the embodiment illustrated in FIGS. 12A and 12B. The adjustable connection is created between a first region 1302 of the gastrointestinal tract and a second region 1304 of the gastrointestinal tract using an adjustable implant 1306. First region 1302 of the gastrointestinal tract fits into an annular cavity located on one side of adjustable implant 1306. Similarly, second region 1304 of the gastrointestinal tract fits into an annular cavity located on the other side of adjustable implant 1306. Adjustable implant 1306 encloses a lumen 1308. The size of lumen 1308 is adjusted by inflation or deflation of an inflatable member 1310. FIG. 13A illustrates a sectional view of the adjustable connection when inflatable member 1310 is in a deflated state. FIG. 13B illustrates a sectional view of the adjustable connection when inflatable member 1310 is in an inflated state.

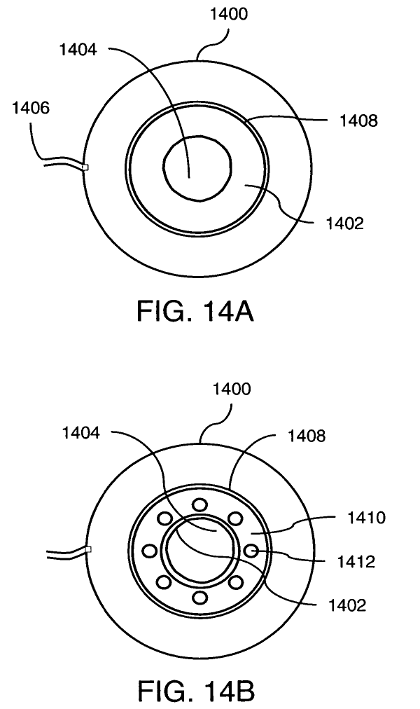

FIGS. 14A and 14B illustrate a fourth embodiment of a device to create an adjustable opening. FIGS. 14A and 14B illustrate two sides of an adjustable implant 1400. Adjustable implant 1400 is substantially ring shaped and comprises an inflatable member 1402. Inflatable member 1402 encloses a lumen 1404. A connection between a first region of the gastrointestinal tract and a second region of gastrointestinal tract is enclosed in lumen 1404. The diameter of the connection is adjusted by inflation or deflation of inflatable member 1402 through a port 1406. Adjustable implant 1400 further comprises a circular flange 1408. Circular flange 1408 is located around lumen 1404 and is present on both sides of adjustable implant 1400. Circular flange 1408 is of a height sufficient to enclose a region around the connection between the first region of the gastrointestinal tract and the second region of the gastrointestinal tract. In one embodiment, the first region of the gastrointestinal tract and the second region of the gastrointestinal tract are both sutured to circular flange 1408. Adjustable implant 1400 further comprises an annular region 1410 comprising apertures 1412. Apertures 1412 enable regions of the gastrointestinal tract to come into physical contact with each other. This facilitates the regions of the gastrointestinal tract to fuse with each other to create a stable anastomosis. In one embodiment, annular region 1410 is biofragmentable or bioabsorbable.

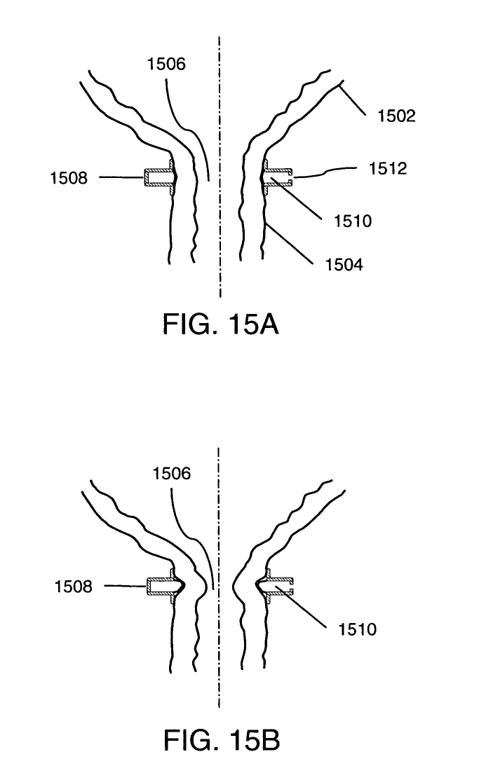

FIGS. 15A and 15B illustrate a sectional view of an adjustable connection made using the embodiment illustrated in FIGS. 14A and 14B. The adjustable connection is created between a first region 1502 of the gastrointestinal tract and a second region 1504 of the gastrointestinal to enclose a lumen 1506. The adjustable connection is enclosed by an adjustable implant 1508. Adjustable implant comprises an inflatable member 1510. The size of lumen 1506 is adjusted by inflation or deflation of inflatable member 1510 through the introduction or removal of an inflating fluid through an inflation port 1512. FIG. 15A illustrates a sectional view of the adjustable connection when inflatable member 1510 is in a deflated state. FIG. 15B illustrates a sectional view of the adjustable connection when inflatable member 1510 is in an inflated state.

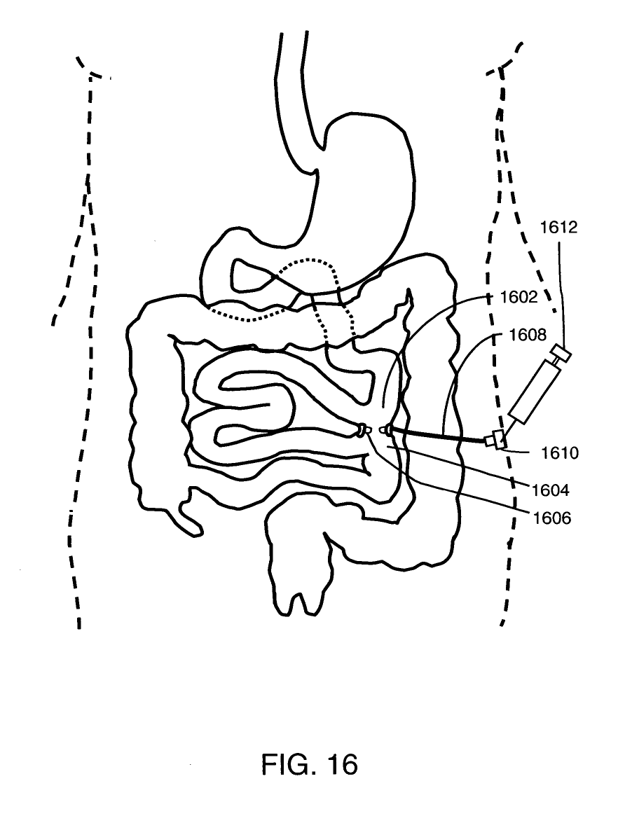

FIG. 16 illustrates an embodiment of a mechanism to adjust the adjustable implant illustrated in FIG. 14A and 14B. In this embodiment, a bypass is created between a first region 1602 of the intestine and a second region 1604 of the intestine using an implant 1606. Implant 1606 comprises an adjustable opening in the form of an inflatable member that is connected to a fluid introducing tube 1608. Fluid introducing tube 1608 is further connected to an injection port 1610. In one embodiment, injection post 1610 is located under the skin such as in the subcutaneous region. The adjustable opening of implant 1606 can be adjusted by introduction or removal of an inflating fluid through injection port 1610 by a syringe 1612.

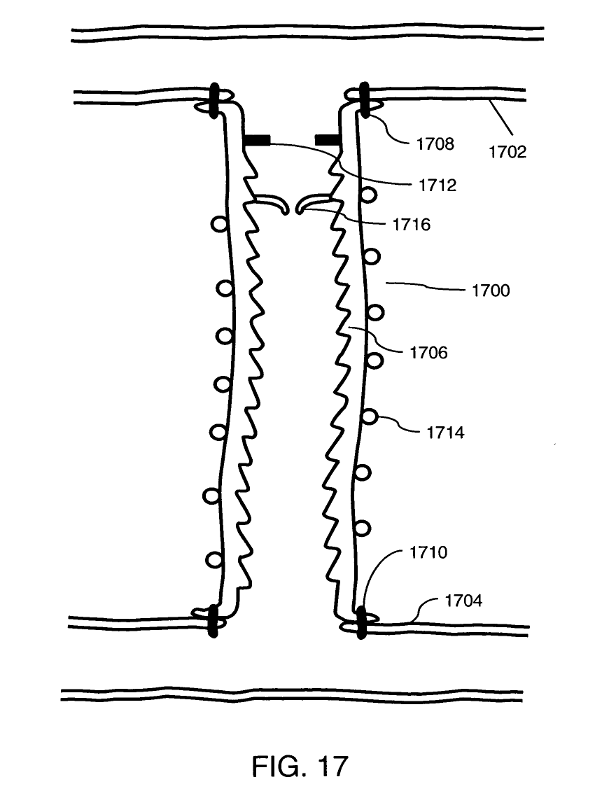

FIG. 17 illustrates a sectional view of a fifth embodiment of the invention. An intestinal bypass graft 1700 used to create a bypass between a first region 1702 of the intestine and a second region 1704 of the intestine. Intestinal bypass graft 1700 comprises a tubular implant 1706. Tubular implant 1706 can be made of suitable biocompatible materials like silicone gel, polyurethane, ultra high molecular weight polyethylene, polyethylene terephthalate, polypropylene, polytetrafloroethylene and polyamides. In one embodiment, the walls of the tubular implant are hollow and are filled with a filler material. Examples of filler material that can be used are silicon gel, saline, soybean oil, hydro gel, polyvinylpyrrolidone, polyethylene glycol, and hyaluronic acid. The inner surface of tubular implant 1706 comprises a series of projections. The projections help the food material in the intestine to flow in a single direction. One end of tubular implant 1706 is connected to first region 1702 of intestine by one or more fasteners 1708 to create an end-to-side anastomosis. Fasteners 1708 are biocompatible. Examples of materials that can be used as fasteners 1708 are sutures, clips, staples, screws, tags and adhesives. The other end of tubular implant 1706 is connected to second region 1704 of intestine by one or more fasteners 1710 to create an end-to-side anastomosis. Fasteners 1710 are biocompatible. Examples of materials that can be used as fasteners 1710 are sutures, clips, staples, screws, tags and adhesives. Tubular implant 1706 is provided with an adjustable opening 1712. Adjustable opening 1712 regulates the amount of food that passes through intestinal bypass graft 1700. Increasing the size of adjustable opening 1712 increases the amount of food passing through intestinal bypass graft 1700. This reduces the amount of consumed food that is absorbed by the patient's body and increases the rate of weight loss. Similarly, reducing the size of adjustable opening 1712 reduces the rate of weight loss. Thus the rate of weight loss can be regulated by changing the size of adjustable opening 1712. Tubular implant 1706 is further provided with an elastic mechanism 1714. Elastic mechanism 1714 provides elasticity to tubular implant 1706. The motion of the patient and the peristaltic motion of the patient's intestines cause various regions of tubular implant 1706 to move with respect to each other. This movement facilitates the flow of food material passing through tubular implant 1706. In one embodiment, elastic mechanism 1714 is in the form of a spring wound around tubular implant 1706. Several biocompatible materials like titanium alloys, stainless steel alloys or elastic biocompatible polymers can be used for constructing the spring. Tubular implant 1706 further comprises a valve 1716. Valve 1716 facilitates the flow of food material in a single direction by preventing backflow of the food material. Valve 1716 can be a mechanical valve or a bioprosthetic valve. Examples of mechanical valves that can be used are ball valves, single-leaflet (tilting disk) valves and bileaflet valves. They can be made of one or more biocompatible materials like collagen, stainless steel, titanium, pyrolytic carbon, Teflon™ or Dacron™. Bioprosthetic valves can be made from animal or human tissues.

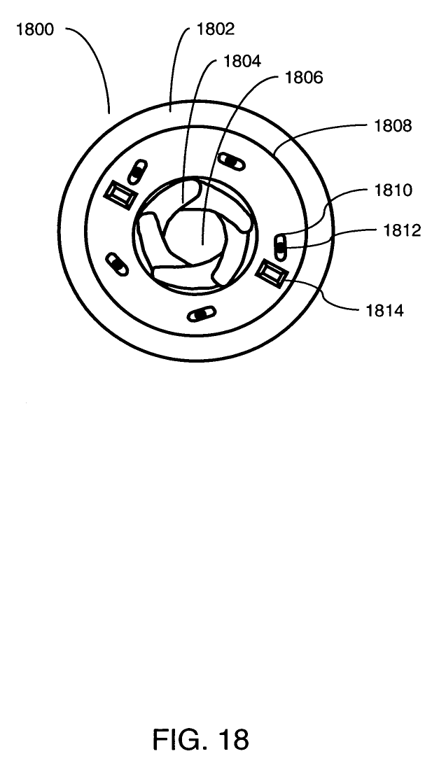

FIG. 18 illustrates a sixth embodiment of the invention. In this embodiment, an adjustable implant 1800 is provided. Adjustable implant 1800 comprises an iris diaphragm. The iris diaphragm comprises a base plate 1802. Base plate 1802 is annular in shape. Adjustable implant 1800 further comprises a plurality of blades 1804. Each blade is attached to base plate 1802 by a pivot in such a way that blades 1804 enclose a lumen 1806. Adjustable implant 1800 further comprises a blade actuating ring 1808 attached coaxially to base plate 1802. Blade actuating ring 1808 can rotate around its axis. Blade actuating ring 1808 is provided with a plurality of slots 1810. The number of slots on blade actuating ring 1808 is equal to the number of blades attached to base plate 1802. Each blade is provided with a projection 1812. Projection 1812 of each blade slides within a slot on blade actuating ring 1808. Thus, each blade is pivoted on base plate 1802 and communicates with blade actuating ring 1808. Blade actuating ring 1808 is further provided with a plurality of gripping slots 1814. Gripping slots 1814 are used to grip and rotate blade actuating ring 1808. Rotation of blade actuating ring 1808 changes the orientation of blades 1804. This changes the size of lumen 1806. Thus, the size of adjustable opening in the invention can be changed by rotating blade actuating ring 1808. In one embodiment, blade actuating ring 1808 is rotated using endoscopic means. Several biocompatible materials like titanium alloys, stainless steel alloys or elastic biocompatible polymers can be used for constructing adjustable implant 1800.

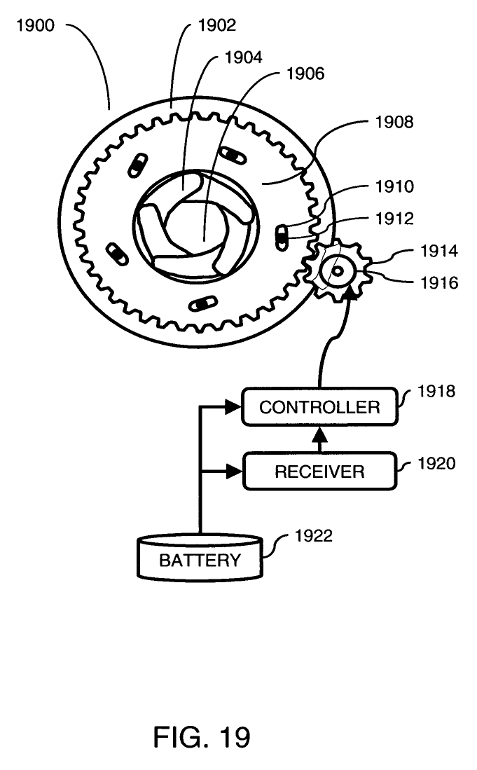

FIG. 19 illustrates a seventh embodiment of the invention. In this embodiment, an adjustable implant 1900 is provided. Adjustable implant 1900 comprises an iris diaphragm that is adjusted using electromagnetic signals. Adjustable implant 1900 comprises a base plate 1902. Base plate 1902 is annular in shape. Adjustable implant 1900 further comprises a plurality of blades 1904. Each blade is attached to base plate 1902 by a pivot in such a way that blades 1904 enclose a lumen 1906. Adjustable implant 1900 further comprises a blade actuating ring 1908 attached coaxially to base plate 1902. Blade actuating ring 1908 can rotate around its axis and can act as a gear. Blade actuating ring 1908 is provided with a plurality of slots 1910. The number of slots on blade actuating ring 1908 is equal to the number of blades attached to base plate 1902. Each blade is provided with a projection 1912. Projection 1912 of each blade slides within a slot on blade actuating ring 1908. Thus, each blade is pivoted on base plate 1902 and communicates with blade actuating ring 1908. Blade actuating ring 1908 is geared to a driver gear 1914. Driver gear 1914 is connected to a control mechanism comprising a motor 1916 and a controller 1918 that supplies a controlled amount of electric current to motor 1916. Controller 1918 is connected to a receiver 1920. Receiver 1920 receives electromagnetic signals and converts the received electromagnetic signals to electric signals and transmits the electric signals to controller 1918. A battery 1922 supplies electric energy to controller 1918 and receiver 1920.

Receiver 1920 receives electromagnetic signals containing information about a required change in size of the adjustable opening. Receiver 1920 converts the electromagnetic signals to electric signals and transmits the electric signals to controller 1918. Controller 1918 calculates the required electric current to cause the required change in size of the adjustable opening. The required electric current is then delivered to motor 1916 causing driver gear 1914 to rotate. Rotation of driver gear 1914 causes blade actuating ring 1908 to rotate. Rotation of blade actuating ring 1908 changes orientation of blades 1904. This changes the size of lumen 1906. Thus, the size of adjustable opening in the invention can be changed. In one embodiment, controller 1918, receiver 1920 and battery 1922 are implanted in the patient's body. In another embodiment, battery 1922 comprises a self-charging mechanism whereby motion of the patient is converted to electrical energy that charges battery 1922. The electromagnetic signals are generated out of the patient's body by an external remote controller. This enables the non-invasive adjustment of adjustable implant 1900. Several biocompatible materials like titanium alloys, stainless steel alloys or elastic biocompatible polymers can be used for constructing the adjustable implant 1900.

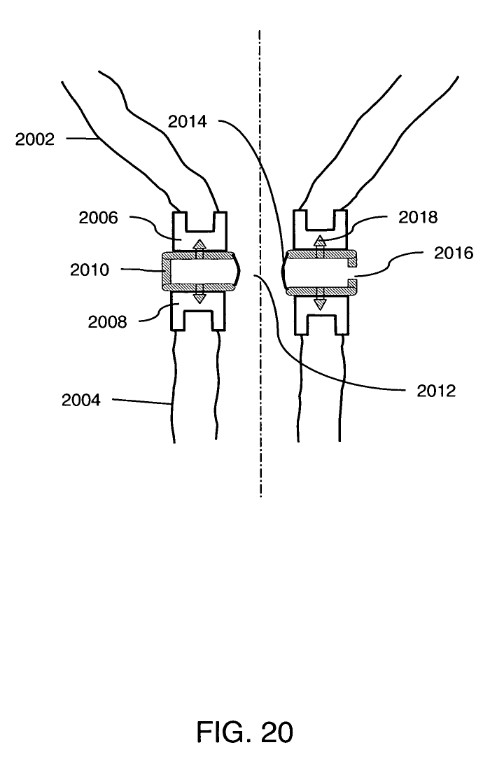

FIG. 20 illustrates a sectional view of an eighth embodiment of the invention. In this embodiment, a gastrointestinal bypass with an adjustable opening is created between a first region 2002 of the gastrointestinal tract and a second region 2004 of the gastrointestinal tract. The adjustable opening is formed by an implant comprising three parts. First part 2006 of the implant is attached to first region 2002 of the gastrointestinal tract. Second part 2008 of the implant is attached to second region 2004 of the gastrointestinal tract. First part 2006 of the implant and second part 2008 of the implant can be attached to the gastrointestinal tract by several methods like suturing, clipping, stapling or using screws, tags or surgical adhesives. Third part 2010 of the implant is attached to first part 2006 of the implant and second part 2008 of the implant. Third part 2010 of the implant encloses a lumen 2012. Third part 2010 of the implant comprises an adjustable opening. In one embodiment, the adjustable opening is in the form of an inflatable member 2014 connected to an inflation port 2016. Inflation or deflation of inflatable member 2014 through inflation port 2016 changes the size of lumen 2012. In this way, the size of the adjustable opening can be adjusted. First part 2006 of the implant, second part 2008 of the implant and third part 2010 of the implant further comprise a locking mechanism 2018 to securely hold together the various parts of the implant.

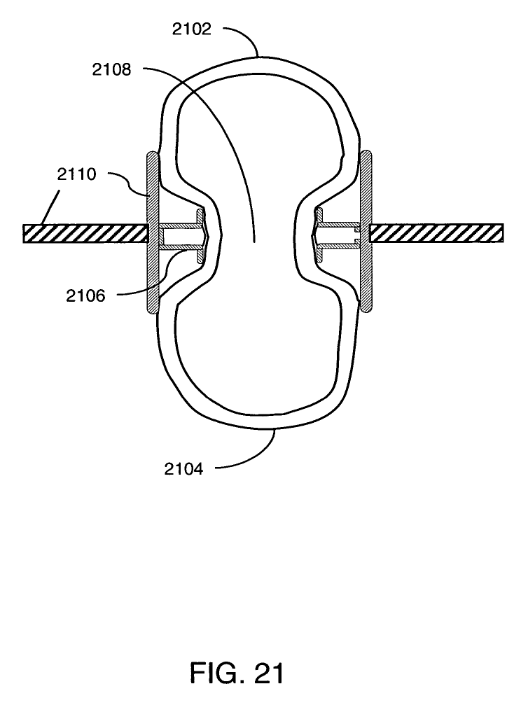

FIG. 21 illustrates a sectional view of a ninth embodiment of the invention. In this embodiment, a side-to-side anastomosis with an adjustable opening is created between a first region 2102 of the gastrointestinal tract and a second region 2104 of the gastrointestinal tract using an adjustable implant 2106. Adjustable implant 2106 can be adjusted to adjust the size of anastomosis lumen 2108 enclosed by adjustable implant 2106. Adjustable implant 2106 further comprises stabilization means 2110 to stabilize the orientation of adjustable implant 2106 with respect to the patient's anatomy. In one embodiment, stabilization means 2110 are in the form of baffles that stabilize the orientation of adjustable implant 2106 with respect to the patient's visceral organs like the intestine.

The described embodiments can be made of suitable biocompatible materials like silicone rubber, polyethylene terephthalate, ultra high molecular weight polyethylene, expanded polytetrafloroethylene, polypropylene, polycarbonate urethane, polyurethane, polyamides, stainless steel 316, titanium, nickel-titanium alloys and cobalt alloys. The described embodiments may comprise a suitable radio-opaque marker for radiographic determination of the position and the level of dilation or contraction of the adjustable implants. The described embodiments may be used as temporary or permanent implants. The embodiments can be used for end-to-end, end-to-side or side-to-side anastomosis. Although the invention is primarily described and illustrated as a gastrointestinal device, it is understood that it can also be used for other anastomosis procedures such as vascular anastomosis.

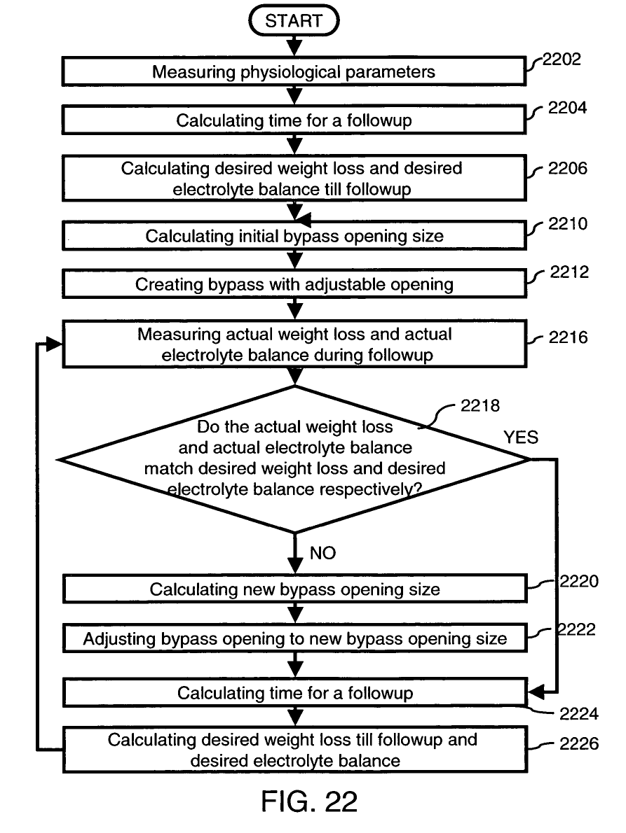

FIG. 22 illustrates the method of the present invention to achieve weight loss in obese patients. The method of the present invention is based on periodically monitoring the patient's physiological parameters and adjusting the size of a gastrointestinal bypass. At step 2202, the patient's initial physiological parameters are measured. Some examples of the physiological parameters that are measured are total weight, body mass index, concentration of blood glucose and electrolyte balance. Electrolyte balance is the balance of physiologically crucial compounds like vitamins, and serum electrolytes such as calcium, magnesium, iron and phosphate. Based on these physiological parameters, at step 2204, a time is fixed for a followup of the patient after the creation of a gastrointestinal bypass. The aim of the followup is to monitor the patient's health status and the effectiveness of the weight loss method. At step 2206, a desired weight loss is calculated based on the patient's physiological parameters. The desired weight loss is in the form of a range of weight loss that is desired in the patient until the followup. Also, at step 2206, a desired electrolyte balance is calculated for the patient. A proper balance of electrolytes such as calcium, magnesium, iron and phosphate and of vitamin D is crucial for the normal functioning of the body. A poorly designed weight loss program can lead to an excessive loss of electrolytes from the body. At step 2210, an initial gastrointestinal bypass opening size is calculated based on the patient's physiological parameters, the desired weight loss and the desired electrolyte balance. At step 2212, a gastrointestinal bypass with an adjustable opening is created in the patient. The initial size of the adjustable opening is the initial gastrointestinal bypass opening size determined at step 2210. Thereafter, the patient is asked to appear for followup at the time calculated at step 2204. During the followup, at step 2216, the patient's actual weight loss and actual electrolyte balance is measured. At step 2218, the desired weight loss and the actual weight loss are compared. Also, at step 2218, the desired electrolyte balance and the actual electrolyte balance are compared. If the desired weight loss and the actual weight loss are not comparable or if the desired electrolyte balance and the actual electrolyte balance are not comparable, the method proceeds to step 2220. At step 2220, a new gastrointestinal bypass opening size is calculated. The calculation is done by taking into consideration the desired weight loss, the actual weight loss, the desired electrolyte balance and the actual electrolyte balance. At step 2222, the intestinal bypass is adjusted to the new bypass opening size calculated at step 2220. At step 2224, a time is fixed for the followup of the patient. At step 2226, a desired weight loss is calculated. The desired weight loss is in the form of a range of weight loss that is desired in the patient until the followup calculated at step 2224. Also, at step 2226, a desired electrolyte balance is calculated for the patient. Thereafter, the method proceeds to step 2216.

Referring back to step 2218, if the desired weight loss and the actual weight loss are comparable and the desired electrolyte balance and the actual electrolyte balance are comparable, the method proceeds to step 2224.

While the preferred embodiments of the invention have been described, it will be clear that the invention is not limited to these embodiments only. Several modifications, changes, variations, substitutions and equivalents will be apparent to persons skilled in the art without departing from the spirit and scope of the invention as described in the claims.

1. An implantable medical device comprising:
an implant defining a lumen that creates a bypass between a first region of the gastrointestinal tract and a second region of the gastrointestinal tract, wherein a fraction of food material passing through the gastrointestinal tract passes through the bypass.

2. The device as recited in claim 1, wherein the first region of the gastrointestinal tract is selected from the group consisting of stomach, small intestine and large intestine.

3. The device as recited in claim 1, wherein the second region of the gastrointestinal tract is selected from the group consisting of stomach, small intestine and large intestine.

4. The device as recited in claim 1, wherein the implant comprises an adjustable opening to adjust the fraction of food material passing through the bypass.

5. The device as recited in claim 4, wherein the adjustable opening can be adjusted by endoscopic means.

6. The device as recited in claim 4, wherein the adjustable opening comprises a deformable element, wherein the size of the adjustable opening is adjusted by deformation of the deformable element.

7. The device as recited in claim 4, wherein the adjustable opening comprises an inflatable element.

8. The device as recited in claim 7, wherein the inflatable element is inflated with a fluid selected from the group consisting of silicon gel, saline, soybean oil, hydro gel, polyvinylpyrrolidone, polyethylene glycol, and hyaluronic acid.

9. The device as recited in claim 7, further comprising an arrangement for introducing or removing fluid from the inflatable element.

10. The device as recited in claim 4, further comprising a system for adjusting the size of the adjustable opening; the system comprising:
a. a receiver for
i. receiving electromagnetic signals from an external source; and
ii. converting the electromagnetic signals to electrical signals;
b. a control mechanism for
i. receiving electrical signals from the receiver; and
ii. adjusting the size of the adjustable opening; and
c. an energy supplying system for supplying energy to the receiver and the control mechanism.

11. The device as recited in claim 1, wherein the implant comprises a valve mechanism that facilitates flow of food material in one direction through the bypass.

12. The device as recited in claim 1, wherein the implant comprises a substantially tubular region.

13. The device as recited in claim 12, wherein the implant comprises an elastic mechanism to facilitate transfer of food material.

14. The device as recited in claim 12, wherein the implant comprises one or more projections on the inner surface of the implant to facilitate transfer of food material in one direction.

15. The device as recited in claim 1, wherein the implant comprises a substantially ring shaped element that creates a direct connection between the first region of the gastrointestinal tract and the second region of the gastrointestinal tract.

16. The device as recited in claim 1, wherein the implant is connected to the gastrointestinal tract by a set of biocompatible fasteners selected from the group consisting of sutures, clips, staples, screws, tags and adhesives.

17. The device as recited in claim 1, wherein the implant comprises:
a. a first element that is attached to the first region of the gastrointestinal tract; and
b. a second element that is attached to the second region of the gastrointestinal tract; wherein the first element and the second element can be attached to each other.

18. The device as recited in claim 17, wherein the implant comprises a third element that is attached between the first element and the second element.

19. The device as recited in claim 1, wherein the implant incorporates an aperture that enables direct physical contact between at least one portion of the first region of the gastrointestinal tract and at least one portion of the second region of the gastrointestinal tract.

20. The device as recited in claim 1, wherein the implant comprises an bioabsorbable element comprising a material selected from the group consisting of polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid) and combinations thereof.

21. The device as recited in claim 1, wherein the implant comprises a material selected from the group consisting of silicone rubber, polyethylene terephthalate, ultra high molecular weight polyethylene, expanded polytetrafloroethylene, polypropylene, polycarbonate urethane, polyurethane, polyamides, stainless steel 316, titanium, a nickel-titanium alloy and a cobalt alloy.

22. The device as recited in claim 1, wherein the implant comprises an impregnated antibiotic.

23. The device as recited in claim 1, wherein the implant comprises a radio-opaque marker.

24. The device as recited in claim 1, wherein the implant comprises a stabilization mechanism to stabilize the orientation of the implant relative to the body's anatomy.

25. The device as recited in claim 1, wherein the lumen cross-section is elongated along one direction.

26. An implantable medical device comprising:
an implant defining a lumen that creates a bypass between a first region of the gastrointestinal tract and a second region of the gastrointestinal tract, wherein a fraction of food material passing through the gastrointestinal tract passes through the bypass; the implant comprising an adjustable opening to adjust the fraction of food material passing through the bypass.

27. The device as recited in claim 26, wherein the first region of the gastrointestinal tract is selected from the group consisting of stomach, small intestine and large intestine.

28. The device as recited in claim 26, wherein the second region of the gastrointestinal tract is selected from the group consisting of stomach, small intestine and large intestine.

29. The device as recited in claim 26, wherein the adjustable opening can be adjusted by endoscopic means.

30. The device as recited in claim 26, wherein the adjustable opening comprises a deformable element, wherein the size of the adjustable opening is adjusted by deformation of the deformable element.

31. The device as recited in claim 26, wherein the adjustable opening comprises an inflatable element.

32. The device as recited in claim 31, wherein the inflatable element is inflated with a fluid selected from the group consisting of silicon gel, saline, soybean oil, hydro gel, polyvinylpyrrolidone, polyethylene glycol, and hyaluronic acid.

33. The device as recited in claim 31, further comprising an arrangement for introducing or removing fluid from the inflatable element.

34. The device as recited in claim 26, further comprising a system for adjusting the size of the adjustable opening; the system comprising:
a. a receiver for
i. receiving electromagnetic signals from an external source; and
ii. converting the electromagnetic signals to electrical signals;
b. a control mechanism for
i. receiving electrical signals from the receiver; and
ii. adjusting the size of the adjustable opening; and
c. an energy supplying system for supplying energy to the receiver and the control mechanism.

35. The device as recited in claim 26, wherein the implant comprises a valve mechanism that facilitates flow of food material in one direction through the bypass.

36. The device as recited in claim 26, wherein the implant comprises a substantially tubular region.

37. The device as recited in claim 36, wherein the implant comprises an elastic mechanism to facilitate transfer of food material.

38. The device as recited in claim 36, wherein the implant comprises one or more projections on the inner surface of the implant to facilitate transfer of food material in one direction.

39. The device as recited in claim 26, wherein the implant comprises a substantially ring shaped element that creates a direct connection between the first region of the gastrointestinal tract and the second region of the gastrointestinal tract.

40. The device as recited in claim 26, wherein the implant is connected to the gastrointestinal tract by a set of biocompatible fasteners selected from the group consisting of sutures, clips, staples, screws, tags and adhesives.

41. The device as recited in claim 26, wherein the implant comprises:
a. a first element that is attached to the first region of the gastrointestinal tract; and
b. a second element that is attached to the second region of the gastrointestinal tract; wherein the first element and the second element can be attached to each other.

42. The device as recited in claim 41, wherein the implant comprises a third element that is attached between the first element and the second element.

43. The device as recited in claim 26, wherein the implant incorporates an aperture that enables direct physical contact between at least one portion of the first region of the gastrointestinal tract and at least one portion of the second region of the gastrointestinal tract.

44. The device as recited in claim 26, wherein the implant comprises an bioabsorbable element comprising a material selected from the group consisting of polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid) and combinations thereof.

45. The device as recited in claim 26, wherein the implant comprises a material selected from the group consisting of silicone rubber, polyethylene terephthalate, ultra high molecular weight polyethylene, expanded polytetratloroethylene, polypropylene, polycarbonate urethane, polyurethane, polyamides, stainless steel 316, titanium, a nickel-titanium alloy and a cobalt alloy.

46. The device as recited in claim 26, wherein the implant comprises an impregnated antibiotic.

47. The device as recited in claim 26, wherein the implant comprises a radio-opaque marker.

48. The device as recited in claim 26, wherein the implant comprises a stabilization mechanism to stabilize the orientation of the implant relative to the body's anatomy.

49. The device as recited in claim 26, wherein the lumen cross-section is elongated along one direction.

50. An anastomosis device comprising:
an implant defining a lumen that creates a bypass between a first region of an anatomical tract and a second region of the anatomical tract, wherein a fraction of material passing through the anatomical tract passes through the bypass.

51. The device as recited in claim 50, wherein the anatomical tract is the gastrointestinal tract.

52. The device as recited in claim 51, wherein the first region of the gastrointestinal tract is selected from the group consisting of stomach, small intestine and large intestine.

53. The device as recited in claim 51, wherein the second region of the gastrointestinal tract is selected from the group consisting of stomach, small intestine and large intestine.

54. The device as recited in claim 50, wherein the implant comprises an adjustable opening to adjust the fraction of material passing through the bypass.

55. The device as recited in claim 54, wherein the adjustable opening can be adjusted by endoscopic means.

56. The device as recited in claim 54, wherein the adjustable opening comprises a deformable element, wherein the size of the adjustable opening is adjusted by deformation of the deformable element.

57. The device as recited in claim 54, wherein the adjustable opening comprises an inflatable element.

58. The device as recited in claim 57, wherein the inflatable element is inflated with a fluid selected from the group consisting of silicon gel, saline, soybean oil, hydro gel, polyvinylpyrrolidone, polyethylene glycol, and hyaluronic acid.

59. The device as recited in claim 57, further comprising an arrangement for introducing or removing fluid from the inflatable element.

60. The device as recited in claim 54, further comprising a system for adjusting the size of the adjustable opening; the system comprising:
a. a receiver for
i. receiving electromagnetic signals from an external source; and
ii. converting the electromagnetic signals to electrical signals;
b. a control mechanism for
i. receiving electrical signals from the receiver; and
ii. adjusting the size of the adjustable opening; and
c. an energy supplying system for supplying energy to the receiver and the control mechanism.

61. The device as recited in claim 50, wherein the implant comprises a valve mechanism that facilitates flow of material in one direction through the bypass.

62. The device as recited in claim 50, wherein the implant comprises a substantially tubular region.

63. The device as recited in claim 62, wherein the implant comprises an elastic mechanism to facilitate transfer of material.

64. The device as recited in claim 62, wherein the implant comprises one or more projections on the inner surface of the implant to facilitate transfer of material in one direction.

65. The device as recited in claim 50, wherein the implant comprises a substantially ring shaped element that creates a direct connection between the first region of the anatomical tract and the second region of the anatomical tract.

66. The device as recited in claim 50, wherein the implant is connected to the anatomical tract by a set of biocompatible fasteners selected from the group consisting of sutures, clips, staples, screws, tags and adhesives.

67. The device as recited in claim 50, wherein the implant comprises:
a. a first element that is attached to the first region of the anatomical tract;
and
b. a second element that is attached to the second region of the anatomical tract; wherein the first element and the second element can be attached to each other.

68. The device as recited in claim 67, wherein the implant comprises a third element that is attached between the first element and the second element.

69. The device as recited in claim 50, wherein the implant incorporates an aperture that enables direct physical contact between at least one portion of the first region of the anatomical tract and at least one portion of the second region of the anatomical tract.

70. The device as recited in claim 50, wherein the implant comprises an bioabsorbable element comprising a material selected from the group consisting of polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid) and combinations thereof.

71. The device as recited in claim 50, wherein the implant comprises a material selected from the group consisting of silicone rubber, polyethylene terephthalate, ultra high molecular weight polyethylene, expanded polytetrafloroethylene, polypropylene, polycarbonate urethane, polyurethane, polyamides, stainless steel 316, titanium, a nickel-titanium alloy and a cobalt alloy.

72. The device as recited in claim 50, wherein the implant comprises an impregnated antibiotic.

73. The device as recited in claim 50, wherein the implant comprises a radio-opaque marker.

74. The device as recited in claim 50, wherein the implant comprises a stabilization mechanism to stabilize the orientation of the implant relative to the body's anatomy.

75. The device as recited in claim 50, wherein the lumen cross-section is elongated along one direction.

76. An anastomosis device comprising:
an implant defining a lumen that creates a bypass between a first region of an anatomical tract and a second region of the anatomical tract, wherein a fraction of material passing through the anatomical tract passes through the bypass; the implant comprising an adjustable opening to adjust the fraction of the material passing through the bypass.

77. The device as recited in claim 76, wherein the anatomical tract is the gastrointestinal tract.

78. The device as recited in claim 77, wherein the first region of the gastrointestinal tract is selected from the group consisting of stomach, small intestine and large intestine.

79. The device as recited in claim 77, wherein the second region of the gastrointestinal tract is selected from the group consisting of stomach, small intestine and large intestine.

80. The device as recited in claim 76, wherein the adjustable opening can be adjusted by endoscopic means.

81. The device as recited in claim 76, wherein the adjustable opening comprises a deformable element, wherein the size of the adjustable opening is adjusted by deformation of the deformable element.

82. The device as recited in claim 76, wherein the adjustable opening comprises an inflatable element.

83. The device as recited in claim 82, wherein the inflatable element is inflated with a fluid selected from the group consisting of silicon gel, saline, soybean oil, hydro gel, polyvinylpyrrolidone, polyethylene glycol, and hyaluronic acid.

84. The device as recited in claim 82, further comprising an arrangement for introducing or removing fluid from the inflatable element.

85. The device as recited in claim 76, further comprising a system for adjusting the size of the adjustable opening; the system comprising:
a. a receiver for
i. receiving electromagnetic signals from an external source; and
ii. converting the electromagnetic signals to electrical signals;
b. a control mechanism for
i. receiving electrical signals from the receiver; and
ii. adjusting the size of the adjustable opening; and
c. an energy supplying system for supplying energy to the receiver and the control mechanism.

86. The device as recited in claim 76, wherein the implant comprises a valve mechanism that facilitates flow of material in one direction through the bypass.

87. The device as recited in claim 76, wherein the implant comprises a substantially tubular region.

88. The device as recited in claim 87, wherein the implant comprises an elastic mechanism to facilitate transfer of food material.

89. The device as recited in claim 87, wherein the implant comprises one or more projections on the inner surface of the implant to facilitate transfer of material in one direction.

90. The device as recited in claim 76, wherein the implant comprises a substantially ring shaped element that creates a direct connection between the first region of the anatomical tract and the second region of the anatomical tract.

91. The device as recited in claim 76, wherein the implant is connected to the anatomical tract by a set of biocompatible fasteners selected from the group consisting of sutures, clips, staples, screws, tags and adhesives.

92. The device as recited in claim 76, wherein the implant comprises:
a. a first element that is attached to the first region of the anatomical tract;
and
b. a second element that is attached to the second region of the anatomical tract; wherein the first element and the second element can be attached to each other.

93. The device as recited in claim 92, wherein the implant comprises a third element that is attached between the first element and the second element.

94. The device as recited in claim 76, wherein the implant incorporates an aperture that enables direct physical contact between at least one portion of the first region of the anatomical tract and at least one portion of the second region of the anatomical tract.

95. The device as recited in claim 76, wherein the implant comprises an bioabsorbable element comprising a material selected from the group consisting of polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid) and combinations thereof.

96. The device as recited in claim 76, wherein the implant comprises a material selected from the group consisting of silicone rubber, polyethylene terephthalate, ultra high molecular weight polyethylene, expanded polytetrafloroethylene, polypropylene, polycarbonate urethane, polyurethane, polyamides, stainless steel 316, titanium, a nickel-titanium alloy and a cobalt alloy.

97. The device as recited in claim 76, wherein the implant comprises an impregnated antibiotic.

98. The device as recited in claim 76, wherein the implant comprises a radio-opaque marker.

99. The device as recited in claim 76, wherein the implant comprises a stabilization mechanism to stabilize the orientation of the implant relative to the body's anatomy.

100. The device as recited in claim 76, wherein the lumen cross-section is elongated along one direction.

101. A medical method comprising the step of:
creating a bypass comprising an adjustable opening between a first region of the gastrointestinal tract and a second region of the gastrointestinal tract, wherein a fraction of food material passing through the gastrointestinal tract passes through the bypass.

102. The method as recited in claim 101, further comprising the step of adjusting the adjustable opening.

103. The method as recited in claim 102, wherein the step of adjusting the adjustable opening is performed after the creation of the bypass.

104. The method as recited in claim 101, wherein the first region of the gastrointestinal tract is selected from the group consisting of stomach, small intestine and large intestine.

105. The method as recited in claim 101, wherein the second region of the gastrointestinal tract is selected from the group consisting of stomach, small intestine and large intestine.

106. The method as recited in claim 101, wherein the method is used in conjunction with an existing weight loss method selected from the group consisting of surgery, diet modification, exercise therapy and pharmacological therapy.

107. A medical method comprising the step of:
creating a bypass between a first region of the gastrointestinal tract and a second region of the gastrointestinal tract, wherein a fraction of food material passing through the gastrointestinal tract passes through the bypass.

108. The method as recited in claim 107, wherein the bypass comprises an adjustable opening.

109. The method as recited in claim 108, further comprising the step of adjusting the adjustable opening.

110. The method as recited in claim 107, wherein the first region of the gastrointestinal tract is selected from the group consisting of stomach, small intestine and large intestine.

111. The method as recited in claim 107, wherein the second region of the gastrointestinal tract is selected from the group consisting of stomach, small intestine and large intestine.

112. The method as recited in claim 107, wherein the method is used in conjunction with an existing weight loss method selected from the group consisting of surgery, diet modification, exercise therapy and pharmacological therapy.

113. A medical method comprising the step of:
creating a bypass between a first region of an anatomical tract and a second region of the anatomical tract, wherein a fraction of material passing through the anatomical tract passes through the bypass; the bypass comprising an adjustable opening to adjust the fraction of the material passing through the bypass.

114. The method as recited in claim 113, further comprising the step of adjusting the adjustable opening.

115. The method as recited in claim 113, wherein the anatomical tract is the gastrointestinal tract.

116. The device as recited in claim 114, wherein the first region of the gastrointestinal tract is selected from the group consisting of stomach, small intestine and large intestine.

117. The device as recited in claim 114, wherein the second region of the gastrointestinal tract is selected from the group consisting of stomach, small intestine and large intestine.

118. The method as recited in claim 114, wherein the method is used in conjunction with an existing weight loss method selected from the group consisting of surgery, diet modification, exercise therapy and pharmacological therapy.
